(19) United States
(12) Patent Application Publication
(10) Pub. No.: US 2003/0055338 A1
(43) Pub. Date: Mar. 20, 2003

(54) Apparatus and methods for ultrasound imaging with positioning of the transducer array

(76) Inventors: Josef Steininger, Vooklamarkt (AU); Helmut Brandl, Pfaffing (AU)

Correspondence Address: Dean D. Small, McAndrews. Held & Malloy, Ltd., 34th Floor, 500 W. Madison Street, Chicago, IL, 60661, US

(21) Appl. No.: 09/954,947

(22) Filed: Sep. 18, 2001

(51) Int. Cl. A61B 8/00

(52) U.S. Cl. 600/459

(57) ABSTRACT

Apparatus and methods for positioning a transducer array within ultrasound probes are disclosed. In one embodiment, the probe is a 3D probe or a 3D real-time probe and comprises a transducer array that can be repositioned or rotated relative to a probe housing to image locations that could not be viewed from the central scan plane of the probe. In some embodiments, a stepper motor is used to position the transducer array. The drive train for positioning the transducer array may further include a gear coupled to the motor by a belt, and a shaft coupling the gear to the transducer array. Repositioning the transducer array within the probe allows for imaging a variety of areas of a patient without moving the probe housing within the patient. Any of the 2D forms of imaging may be performed at any of the planes within a range of the 3D volume swept by a probe such as a 3D probe or 3D real-time probe in accordance with an embodiment of the present invention.

### BACKGROUND OF THE INVENTION

Aspects of the present invention are directed to the field of ultrasound imaging. More particularly, aspects of the present invention are directed to methods and apparatus for positioning the transducer array within a probe to obtain 2D images.

Medical diagnostic imaging systems exist for many applications, wherein a physician examines a patient by placing an ultrasound probe in an area of interest and examining ultrasound images and the like. In endosonographic applications, the probe must be used in very restricted spaces. Conventional ultrasound probes are capable of scanning a patient area of interest within a thin 2-dimensional scan plane extending away from the probe at an angle perpendicular to the surface of the transducer array within the probe. The scan plane is perpendicular to the transducer array. During an examination, it is desirable to image a patient area of interest at various surfaces and at different angles. Hence, the probe must be moved, reoriented and rotated during an exam to shift the scan plane as desired. Conventional probes for endosonographic applications afford very little freedom of movement because the entire probe would have to be moved. The restriction of movement limits the patient area that may be examined by a conventional probe. In order to view over a greater area, the probe must be moved, often to a position that is uncomfortable for the patient. Thus there is a need to increase the ability to examine volumes in cavities without substantially moving the probe.

Difficult areas to examine when employing probes for endosonographic applications include vaginal cavities and the ovaries, rectal areas, seminal vesicles, the bladder neck, the bladder floor, the bladder triangle, and the base of the prostate. Similarly, it may be difficult to image the parotid glands, the lingual gland, the mouth floor, and the lymph nodes using conventional probes. Some of the foregoing areas are difficult to examine with endosonographic probes because it is difficult for an operator to move the probe without causing patient discomfort. Some areas cannot be reached because the probe cannot be tilted enough to examine those areas. Thus there is a need for a probe that can examine a variety of areas without causing patient discomfort.

### SUMMARY OF THE INVENTION

In accordance with an embodiment of the present invention, a probe comprising a transducer array in a housing is provided. The transducer array is physically movable to be repositioned in the probe housing in order to reorient the scan plane without moving the probe housing. A manual mechanism may be provided for repositioning the transducer array and scan plane. Alternatively, movement of the transducer array may be controlled and caused by integrated probe mechanics, a motor, and motor control. The motor control may be part of the system software. In certain embodiments, the user repositions the transducer array in pre-defined angular increments with a rotational control button.

One embodiment of the present invention comprises an ultrasonic probe for obtaining ultrasound information of a region of interest (ROI), the probe comprising a housing having a central scan plane, a transducer array pivotally mounted within the housing, the transducer array being pivotal around a rotation axis, and a control member pivoting the transducer array about the rotation axis with respect to the central scan plane, the transducer array being configured to transmit and receive ultrasound signals to and from an oblique scan plane oriented at an angle with respect to the central scan plane. The control member may comprise a stepper motor disposed in the housing. The control member may comprise a handcrank. The ultrasonic probe may comprise a position sensing device for sensing an angular position of the transducer array with respect to a reference angle. The ultrasonic probe may comprise a centering device determining when the transducer array is aligned with the central scan plane. The ultrasonic probe may be a rectal probe, an endovaginal probe, a small part probe producing a sector-shaped scan plane, or a small linear probe producing a rectangular-shaped scan plane.

In a further embodiment of the present invention, a method for obtaining 2D images of a region of interest (ROI) comprises the steps of providing a housing having a central scan plane, mounting a transducer array for pivotal motion around a rotation axis, and pivoting the transducer array around the rotation axis with respect to the central scan plane, the transducer array being configured to transmit and receive ultrasound signals to and from an oblique scan plane oriented at an angle with respect to the central scan plane. The method may comprise the step of providing a stepper motor disposed in the housing. The method may comprise the step of providing a handcrank. The method may comprise the step of providing a position sensing device for sensing an angular position of the transducer array with respect to a reference angle. The method may comprise the step of providing a centering device determining when the transducer array is aligned with the central scan plane. The methods may be used with rectal probes, endovaginal probes, small part probes producing a sector-shaped scan plane, or small linear probes producing a rectangular-shaped scan plane.

### BRIEF DESCRIPTION OF THE DRAWINGS

The foregoing summary, as well as the following detailed description of the preferred embodiments of the present invention, will be better understood when read in conjunction with the appended drawings. For the purpose of illustrating the preferred embodiments of the present invention, there is shown in the drawings, embodiments which are presently preferred. It should be understood, however, that the present invention is not limited to the arrangements and instrumentality shown in the attached drawings.

FIG. 1 is a perspective view of a probe formed in accordance with an embodiment of the present invention with portions broke away to reveal structure inside the housing.

FIG. 2 is a perspective view of a portion of a probe formed in accordance with an embodiment of the present invention and depicting an oblique scan plane position.

FIG. 3 is a sectional view of a magnetic centering device used in a probe, formed in accordance with an embodiment of the present invention.

FIG. 4 is a side elevational view of a probe formed in accordance with an embodiment of the present invention having a mechanical mechanism to reposition the scan plane.

FIG. 5 is a block diagram of a rotation control system formed in accordance with an embodiment of the present invention.

FIG. 6 is an illustration of an ultrasound display showing the angular position of a transducer array in accordance with an embodiment of the present invention and a schematic of a scan produced by the transducer array at that position; and

FIG. 7 is an illustration of a display showing the angular position of the transducer array of FIG. 6 and a schematic of the scan produced by the transducer array at the position of the array in FIG. 7, the angular position of the transducer array being different from the angular position shown in FIG. 6.

### DETAILED DESCRIPTION OF THE DRAWINGS

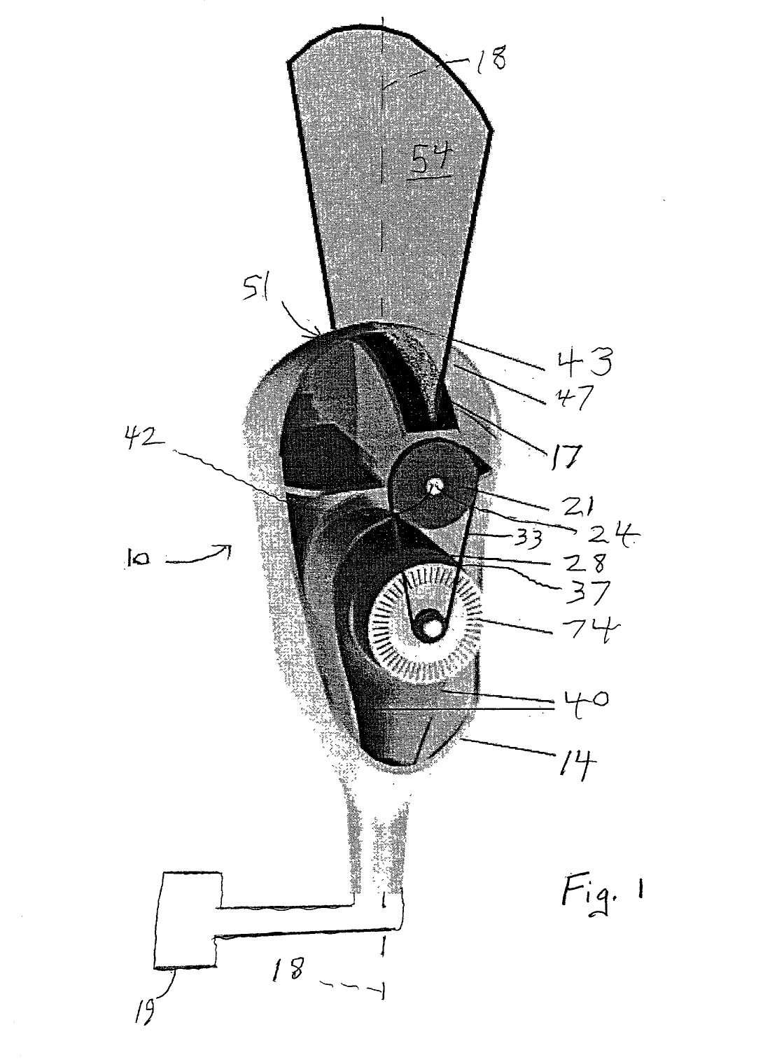

An embodiment of the present invention is shown in FIG. 1 and comprises a scan probe 10, such as a 3D probe or a 3D real-time probe, having a housing 14 and a transducer array 17 in the housing 14. The transducer array 17 may be a one-dimensional array formed from a row of elements, or a two-dimensional array formed from rows and columns of elements when a 2D array is used, it may be electronically focused in the transverse direction. The housing 14 defines a longitudinal axis 18. The housing includes a cavity in which the transducer array 17 is mounted. The cavity is sufficiently large and appropriately shaped to permit arcuate movement of the transducer array 17, such as along an arc of 135 degrees. The size of the arc may depend on the application for which the scan probe 10 is intended. An ultrasound system 19, shown schematically, controls the transducer array 17. The transducer array 17 may be rectangularly shaped and includes one end mounted to a drive shaft 24. The drive shaft 24 pivotally supports the transducer array 17 to enable movement of the transducer array 17 laterally (with respect to a longitudinal axis of the array) along an arc. The drive shaft 24 includes an outer end to which a drive gear 21 is mounted. The gear 21 is coupled to a motor 28 by a drive belt 33. The motor 28 may be a stepper motor 37, which allows precise increments of rotation, enabling an operator to position the transducer array 17 at any desired angle. 3D software for the system and motor control may be used to control the movement of the motor 28.

A transducer cable 40 extends from the array to an interconnect (not seen in FIG. 1) located behind the motor 28. A system cable (also not seen in FIG. 1) extends from the interconnect to the ultrasound system 19 shown schematically in FIG. 1. The system cable is coaxial and comprises a plurality of cables. One of the cables in the system cable provides power to the motor 28.

When the motor 28 is operated, the drive belt 33 is rotated and the gear 21 is turned. The rotating gear 21 rotates the drive shaft 24 that rotates the transducer array 17. More generally, the transducer array 17 is mounted on a mechanism, such as the drive shaft 24, that defines a pivotable axis 42 around which the transducer array 17 may oscillate.

The housing 14 includes an acoustic window portion 43 that allows ultrasound beams and echoes to pass through the housing 14 at that location. The window portion 43 spans an entire range of motion of the transducer array 17. Coupling fluid 47 is positioned between the transducer array 17 and the interior surface of the scan probe 10. The coupling fluid 47 provides special acoustic impedance from the surface of the transducer array 17 to the body of the patient to improve image quality. The transducer array 17 oscillates when driven by the stepper motor 37 and the gear 21. The drive shaft 24, driven by the stepper motor 37, thus moves the transducer array 17 along an arc as the drive shaft 24 rotates around the pivotal axis 42.

The transducer array 17 is relatively small and curved, and oscillates around a central portion 51 of the scan probe 10 which is very close to the inside tip of the scan probe 10. The transducer array 17 has a rotational arc that allows a wide field sweep angle and results in an increase in the area viewed with the transducer array 17. In a rectal probe, for example, the sweep angle range may be 67 degrees each way from a zero or center line. Other probes for other applications may have different ranges of sweep angles. Also, rectal probes may have sweep angle ranges other than 67 degrees.

To acquire a 2D image, the transducer array 17 is rotated to a position within the available field of sweep angles corresponding to a position at which an operator desires to obtain an image. Once the transducer array 17 has been rotated to a desired scan plane, the transducer array 17 is held at the desired scan plane while images are obtained. A series of ultrasound firings are electronically focused at various points and depths along the desired scan plane to obtain echo information throughout the scan plane (or a desired portion thereof).

The scan probe 10 may be held stationary at one angle relative to a patient, while the transducer array 17 moves the scan plane away from a central scan plane 54 of the scan probe 10. Hence, the scan plane is positionable at an oblique angle with respect to the central scan plane 54 and scan probe 10. The central scan plane 54 is substantially parallel to, and includes, the pivotal axis 42 and the longitudinal axis 18 of the housing 14. The pivotal axis 42 and longitudinal axis 18 are oriented co-planar with the central scan plane 54 in FIG. 1, but need not be. The scan probe 10 enables an operator to use a 3D or a 3D real-time (i.e., a 4D) transducer to perform a 2D scan along an angle that is oblique relative to the central scan plane 54 of the scan probe 10.

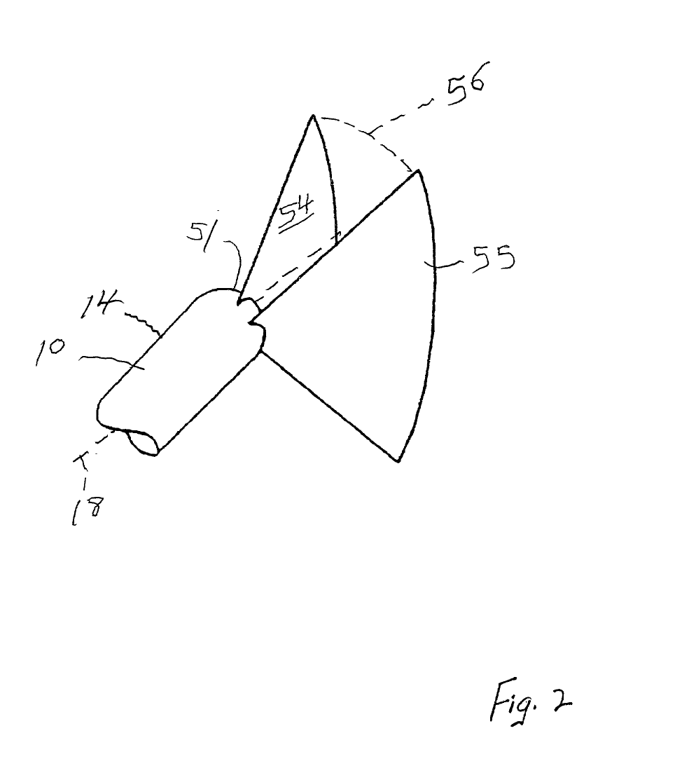

FIG. 2 shows a scan probe 10 formed in accordance with an embodiment of the present invention and illustrates an exemplary scan plane arranged at an oblique angle with respect to the central scan plane 54. The oblique scan plane 55 is illustrated, however any number of other scan planes may be achieved on either side of the central scan plane 54, limited only by the sweep range of transducer array 17. The capability of positioning the transducer array 17 to produce oblique scan plane 55 with respect to the central scan plane 54 is advantageous with angle cavitary examinations in which it is difficult to move the scan probe 10 at significant angles. For example, an operator using a scan probe 10 simply reorients the transducer array 17 relative to the housing 14 without moving the scan probe 10, such as to view a particular ovary.

The stepper motor 37 may be used to help operators keep track of the position of the transducer array 17. Each step of the motor 37 moves the transducer array 17 by the same incremental sweep angle. The stepper motor 37 indicates to an operator the number of steps that have been made. Thus, an operator may simply use the count of steps in each direction, supplied by the stepper motor 37, to determine the net position of the transducer array 17. An equal number of steps away from and back toward a central position will return the transducer array 17 to the central position. Optionally, the system may count steps automatically and display information indicative of the present sweep angular position.

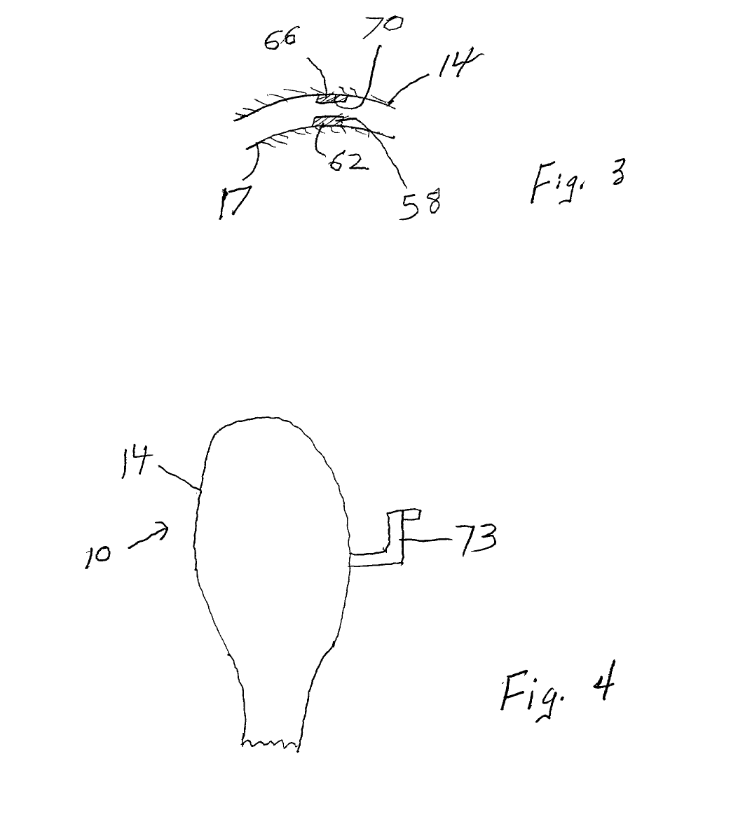

Additionally or alternatively to the stepper motor 37, a first magnet 58 may be placed at a center 62 of the transducer array 17, as shown in FIG. 3, and a Hall sensor 66 may be positioned at a central interior point 70 of the housing 14. The first magnet 58 can be used to determine when the transducer array 17 is at a central position and when the transducer array 17 has been returned to the central position following scans at oblique positions. At the central position, the first magnet 58 and the Hall sensor 66 interact. When the magnet 58 and Hall sensor 66 interact, the system determines that transducer array 17 is in a predefined angular position. Optionally, multiple magnets may be dispersed along the transducer array 17 and/or housing 14.

Other position detection devices may be used instead of magnets. For example, as seen in FIG. 4, a mechanical member, such as a hand crank 73, may be employed to move the transducer array. In such an embodiment, the operator may be able to determine the position of the transducer array 17 by the position of the hand crank 73. An alternative position sensing device is an optical device 74 such as shown in FIG. 1. The optical device 74 includes a series of radial slots or reflective strips evenly spaced about a perimeter of a wheel and at known angular positions. A light source and detector are positioned on opposite sides of the wheel when slots are used and on the same side when reflective strips are used. The detector detects light passed through or reflected from the slots or, reflective strips. The optical device 74 detects the position of the array at all oblique positions. A position detection system such as the optical device 74 is a closed loop mechanism that determines the absolute position of the transducer array 17. Thus, a stepper motor is not needed for determining position when the optical device 74 is employed.

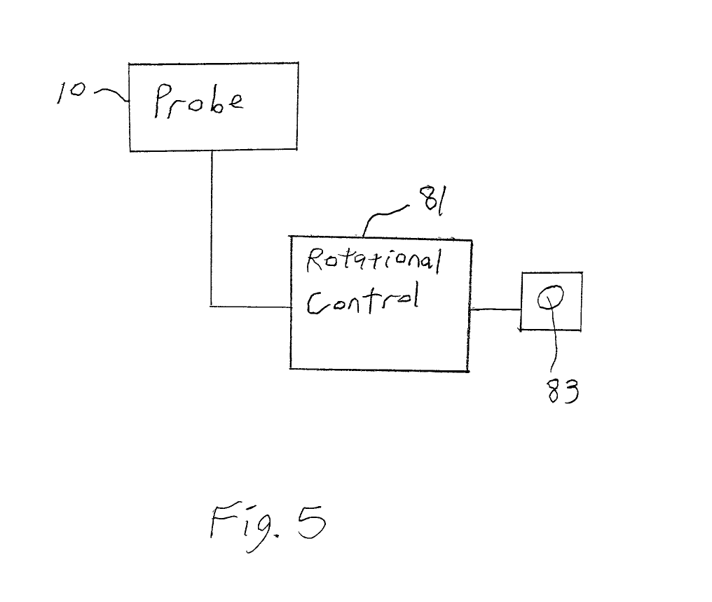

The scan probe 10 may comprise a rotational control device 81, as shown in FIG. 5. The device 81 enables an operator to sweep the transducer array 17 a pre-determined angle simply by pressing a button 83 or sliding a switch. The predetermined angle may be determined by the operator and may vary depending upon the application. The rotational control device 81 is both quick and accurate, and may be used in conjunction with the stepper motor 37 or with other types of motors.

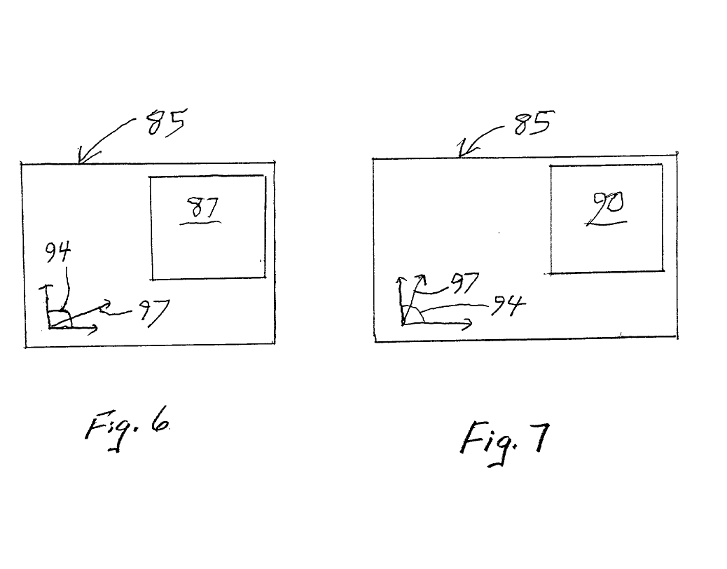

FIGS. 6 and 7 each show an ultrasound display 85 having a B-mode image 87, 90, respectively, of the same patient. The B-mode images 87, 90 are shown schematically in FIGS. 6 and 7. The B-mode images 87, 90 were obtained from the same scan probe 10, but with the transducer array 17 oriented at different angular positions within the patient. The lower left corner of FIGS. 6 and 7 depict a maximum sweep angle 94, and a line 97 indicating the angular position of the transducer array 17 when the respective B-mode image 87 or 90 was taken. The line 97 indicates an exemplary angular position 56 of the transducer array 17 within the possible maximum volume sweep angle 94. The display 85 shown in FIGS. 6 and 7 allows an operator to quickly observe a B-mode image and the angular position 56 of the transducer array 17 from which the B-mode image was obtained by looking at a single display.

By oscillating the transducer array 17, scans of a plurality of areas are possible with no extra discomfort to the patient. For example, the transducer array 17 may be easily swept to a different position starting from the apex or lower portion of the prostate to the base and further to the bladder neck and backwards.

Embodiments of the present invention allow volume sweeps to be performed while activating any normal mode, such as B-mode, harmonic imaging, 2D compounding, and the like. Also, spectral Doppler, color Doppler, or power Doppler can be activated while volume sweeping.

While the invention has been described with reference to a preferred embodiment, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

What is claimed is:

1. An ultrasonic probe for obtaining ultrasound information of a region of interest (ROI), the probe comprising:
a housing having a central scan plane;
a transducer array pivotally mounted within the housing, the transducer array being pivotal around a rotation axis; and
a control member pivoting the transducer array about the rotation axis with respect to the central scan plane, the transducer array being configured to transmit and receive ultrasound signals to and from an oblique scan plane oriented at an angle with respect to the central scan plane.

2. The ultrasonic probe of claim 1, wherein the control member comprises a stepper motor disposed in the housing.

3. The ultrasonic probe of claim 2, and comprising a gear and a belt, wherein the belt couples the gear to the stepper motor.

4. The ultrasonic probe of claim 1, wherein the control member comprises a handcrank.

5. The ultrasonic probe of claim 1, further comprising a position sensing device for sensing an angular position of the transducer array with respect to a reference angle.

6. The ultrasonic probe of claim 1, further comprising an optical sensing device for sensing an angular position of the transducer array with respect to a reference angle.

7. The ultrasonic probe of claim 1, further comprising a centering device determining when the transducer array is aligned with the central scan plane.

8. The ultrasonic probe of claim 7, wherein the centering device is a magnetic sensing device.

9. The ultrasonic probe of claim 1, wherein the probe is configured to obtain 3D volumes of scan planes.

10. The ultrasonic probe of claim 1, further comprising a button directing the control member to rotate the transducer array a predetermined number of degrees each time the button is pressed.

11. The ultrasonic probe of claim 1, further comprising a button directing the control member to rotate the transducer array to a predetermined position relative to the central scan plane.

12. The ultrasonic probe of claim 1, wherein the probe is one of a rectal probe, an endovaginal probe, a small part probe producing a sector-shaped scan plane, and a small linear probe producing a rectangular-shaped scan plane.

13. An ultrasonic probe for obtaining ultrasound information of a region of interest (ROI), the probe comprising:
a housing having a central scan plane;
a transducer array pivotally mounted within the housing, the transducer array being pivotal around a rotation axis; and
a motor pivoting the transducer array about the rotation axis with respect to the central scan plane, the transducer array being configured to transmit and receive ultrasound signals to and from an oblique scan plane oriented at an angle with respect to the central scan plane.

14. The ultrasonic probe of claim 13, wherein the probe is one of a rectal probe, an endovaginal probe, a small part probe producing a sector-shaped scan plane, and a small linear probe producing a rectangular-shaped scan plane.

15. The ultrasonic probe of claim 13, wherein the motor is a stepper motor disposed in the housing.

16. The ultrasonic probe of claim 15, and comprising a gear, attached to the transducer array, and a belt, wherein the belt couples the gear to the stepper motor,

17. The ultrasonic probe of claim 13, further comprising a position sensing device for sensing an angular position of the transducer array with respect to a reference angle.

18. The ultrasonic probe of claim 13, further comprising an optical sensing device for sensing an angular position of the transducer array with respect to a reference angle.

19. The ultrasonic probe of claim 13, further comprising a centering device for determining when the transducer array is aligned with the central scan plane.

20. The ultrasonic probe of claim 19, wherein the centering device is magnetic sensor device.

21. A method for obtaining 2D images of a region of interest (ROI), the method comprising the steps of:
providing a housing having a central scan plane;
mounting a transducer array for pivotal motion around a rotation axis; and
pivoting the transducer array around the rotation axis with respect to the central scan plane, the transducer array being configured to transmit and receive ultrasound signals to and from an oblique scan plane oriented at an angle with respect to the central scan plane.

22. The method of claim 21, and comprising the step of providing a stepper motor disposed in the housing.

23. The method of claim 22, and comprising the step of providing a gear and a belt, wherein the belt couples the gear to the stepper motor.

24. The method of claim 21, further comprising the step of providing a handcrank.

25. The method of claim 21, further comprising the step of providing a position sensing device for sensing an angular position of the transducer array with respect to a reference angle.

26. The method of claim 21, further comprising the step of providing an optical sensing device for sensing an angular position of the transducer array with respect to a reference angle.

27. The method of claim 21, further comprising the step of providing a centering device determining when the transducer array is aligned with the central scan plane.

28. The method of claim 27, wherein the centering device is a magnetic sensoring device.

29. The method of claim 21, wherein the probe is configured to obtain 3D volumes of scan planes.

30. The method of claim 21, farther comprising the step of providing a button for rotating the transducer array a predetermined number of degrees each time the button is pressed.

31. The method of claim 21 and comprising the step of providing a button for rotating the transducer array to a predetermined position relative to the central plane.

32. The method of claim 21 wherein the probe is one of a rectal probe, an endovaginal probe, a small part probe producing a sector-shaped scan plane, or a small linear probe producing a rectangular-shaped scan plane.
